(19) United States
(12) Patent Application Publication
(10) Pub. No.: US 2009/0175795 A1
(43) Pub. Date: Jul. 9, 2009

(54) CHIMERIC THERAPEUTIC AGENTS

(75) Inventor: Mizhou Hui, Camarillo, CA (US)

Correspondence Address: OCCHIUTI ROHLICEK & TSAO, LLP, 10 FAWCETT STREET, CAMBRIDGE, MA, 02138, US

(73) Assignee: Amprotein Corporation, Camarillo, CA (US)

(21) Appl. No.: 11/996,816

(22) PCT Filed: Mar. 28, 2006
(86) PCT No.: PCT/US06/11276

§ 371 Date: Sep. 29, 2008

Related U.S. Application Data
(60) Provisional application No. 60/703,950, filed on Jul. 29, 2005.
(60) Provisional application No. 60/727,612, filed on Oct. 17, 2005.
(60) Provisional application No. 60/762,820, filed on Jan. 27, 2006.
(60) Provisional application No. 60/773,385, filed on Feb. 14, 2006.

(51) Int. Cl. A61K 49/00; C07K 19/00; C07H 21/00; C12N 15/63; C12N 5/10; C12P 21/02; A61K 38/22; A61K 31/7088; A61P 3/10

(52) U.S. Cl. 424/9.2; 530/399; 530/391.7; 536/23.4; 435/320.1; 435/325; 435/69.1; 514/12; 514/44

(57) ABSTRACT

A fusion protein including (i) a first segment that is located at the amino terminus of the fusion protein and contains the sequence of a first biological active peptide or protein; and (ii) a second segment that is located at the carboxyl terminus of the fusion protein and contains the sequence of a second biological active peptide or protein. The first and second segments are operably and covalently linked. Also disclosed are nucleic acids encoding the fusion protein, vectors and host cells having the nucleic acids, and related composition and methods of treating diabetes or/and obesity.

### RELATED APPLICATION

This application claims priority to U.S. provisional Application Serial No. U.S. 60/703,950, filed on Jul. 29, 2005; U.S. provisional Application Serial No. U.S. 60/727,612, filed on Oct. 17, 2005; U.S. provisional Application Serial No. U.S. 60/762,820, filed on Jan. 27, 2006; and U.S. provisional Application Serial No. U.S. 60/773,385 filed on Feb. 14, 2006, the contents of which are incorporated by reference in their entirety.

### BACKGROUND OF THE INVENTION

Diabetes mellitus, commonly called diabetes, refers to a disease process derived from multiple causative factors and characterized by elevated levels of plasma glucose, referred to as hyperglycemia. See, e.g., LeRoith, D. et al., (eds.), DIABETES MELLITUS (Lippincott-Raven Publishers, Philadelphia, Pa. U.S.A. 1996). According to the American Diabetes Association, diabetes mellitus is estimated to affect approximately 6% of the world population. Uncontrolled hyperglycemia is associated with increased and premature mortality due to an increased risk for microvascular and macrovascular diseases, including nephropathy, neuropathy, retinopathy, hypertension, cerebrovascular disease and coronary heart disease. Therefore, control of glucose homeostasis is an important approach for the treatment of diabetes.

There are two major forms of diabetes: Type 1 diabetes (formerly referred to as insulin-dependent diabetes or IDDM); and Type 2 diabetes (formerly referred to as noninsulin dependent diabetes or NIDDM). Type 1 diabetes is the result of an absolute deficiency of insulin, the hormone which regulates glucose utilization. This insulin deficiency is usually characterized by β-cell destruction within the Islets of Langerhans in the pancreas and absolute insulin deficiency. Type 2 diabetes is a disease characterized by insulin resistance accompanied by relative, rather than absolute, insulin deficiency. Type 2 diabetes can range from predominant insulin resistance with relative insulin deficiency to predominant insulin deficiency with some insulin resistance. Insulin resistance is the diminished ability of insulin to exert its biological action across a broad range of concentrations. In insulin resistant individuals the body secretes abnormally high amounts of insulin to compensate for this defect. When inadequate amounts of insulin are present to compensate for insulin resistance and adequately control glucose, a state of impaired glucose tolerance develops. In a significant number of individuals, insulin secretion declines further and the plasma glucose level rises, resulting in the clinical state of diabetes.

The majority of Type 2 diabetic patients are treated either with hypoglycemic agents which act by stimulating release of insulin from beta cells, or with agents that enhance the tissue sensitivity of the patients towards insulin, or with insulin. However, this therapy is, in most instances, not satisfactory.

Insulin stimulates glucose uptake by skeletal muscle and adipose tissues primarily through translocation of the glucose transporter 4 (GLUT4) from the intracellular storage sites of the cell surface (Saltiel, A. R. & Kahn, C. R. (2001) Nature 414:799-806; Saltiel, A. & Pessin, J. E. (2002) Trends in Cell Biol. 12:65-71; White, M. F. (1998) Mol. Cell. Biochem. 182:3-11). In response to insulin, a fraction of GLUT4 present in intracellular membranes is redistributed to the plasma membrane resulting in an increase of GLUT4 on the cell surface and enhanced glucose uptake by these cells. GLUT4 translocation is primarily mediated through the insulin receptor (IR).

In addition to glucose transport, insulin is intimately involved in adipogenesis, a process which involves proliferation of preadipocytes (pre-fat cells) and differentiation of preadipocytes into adipocytes (fat cells) with accumulation of fat in adipocytes. Studies with the adipocyte cell line 3T3-L1 suggest that the role insulin plays in adipogenesis is primarily mitotic. Before differentiation, 3T3-L1 cells are fibroblast-like preadipocytes that contain more IGF-1 receptors than IR. In vitro, adipogenesis of preadipocytes can be triggered by a commonly used differentiation-inducing cocktail, MDI, which consists of an agent methylisobutylxanthine (MIX) that elevates cAMP; a glucocorticoid, dexamethasone (DEX); and insulin (or IGF-1) that interacts with the IGF-1 receptors on the preadipocytes (Tong, Q., Hotamisligil, G. S. (2001) Rev. in Endoc. & Metabolic Disorders. 2:349-355; Rosen, E. D., et al. (2000) Genes Dev. 14:1293-1307). When treated with MDI, confluent preadipocytes re-enter the cell cycle and undergo approximately two rounds of mitosis (Modan-Moses, D., et al. (1998) Biochem. J. 333:825-831; Tong, Q., Hotamisligil, G. S. (2001) Rev. in Endoc. & Metabolic Disorders. 2:349-355; Rosen, E. D., et al. (2000) Genes Dev. 14:1293-1307), a process commonly referred to as clonal expansion. Following clonal expansion, the preadipocytes exit the cell cycle and begin to differentiate into adipocytes by expressing adipocyte genes.

Due to its adipogenic effect, insulin has the undesirable effect of promoting obesity in patients with type 2 diabetes (Moller, D. E. (2001) Nature 414:821-827). Unfortunately, other anti-diabetic drugs which are currently being used to stimulate glucose transport in patients with type 2 diabetes also possess adipogenic activity. Thus while current drug therapy may provide reduction in blood sugar, it often promotes obesity.

Accordingly, it is highly desirable to develop a new generation of anti-diabetic drugs that correct hyperglycemia without generating concomitant adipogenic side effects. Compounds that induce glucose uptake in a diabetic patient without causing hypoglycemia are also desirable. A number of therapeutic proteins have developed for treating diabetes or obesity. However, many of them are not satisfactory due to poor efficacy, side effects, or instability.

On other hand, obesity is a significantly fast-growing human disease in the world. Obese patients often have impaired function of glucose tolerance or “chemical or pre-clinical” diabetes. It is highly desirable to develop a new generation of anti-obesity drugs that correct the impaired function of glucose tolerance. Ideally, compounds that induce weight loss and correct the impaired glucose tolerance in obese patients without causing hypoglycemia are also desirable.

### SUMMARY

This invention relates to using human leptin as a functional fusion partner to extend biological life of anti-diabetes or anti-obesity therapeutic peptides such as glucagon-like peptide -1 (GLP-1) or its analogues, peptide YY, and amylin. The fusion of leptin extends the biological life of the therapeutic peptides and acts in more than additive effect or synergy with the therapeutic peptides.

Accordingly, one aspect of this invention features a fusion protein that includes (i) a first segment that is located at the amino terminus of the fusion protein and contains the sequence of a first biological active peptide or protein; and (ii) a second segment that is located at the carboxyl terminus of the fusion protein and contains the sequence of a second biological active peptide or protein. The first and second segments are operably and covalently linked.

An isolated protein or polypeptide refers to a protein or polypeptide substantially free from naturally associated molecules, i.e., it is at least 75% (i.e., any number between 75% and 100%, inclusive) pure by dry weight. Purity can be measured by any appropriate standard method, e.g., by column chromatography, polyacrylamide gel electrophoresis, or HPLC. An isolated polypeptide of the invention can be purified from a natural source (for wild type polypeptides), produced by recombinant DNA techniques, or by chemical methods.

The first or second biological active peptide or protein can be a peptide hormone or a protein hormone. The first biological active protein can contain the sequence of Glucagon-like peptide 1, amylin, or peptide YY, or a functional equivalent thereof.

In one embodiment, the first biological active protein contains the sequence of SEQ ID NO: 2. The second biological active protein can contain the sequence of Leptin or a functional equivalent or a weight loss inducing protein. It maintains its biological active protein functions when covalently fused to the C-terminus of a heterologous peptide or protein. The second biological active protein contains the sequence of SEQ ID NO: 1. In one example, the fusion protein contains the sequence of SEQ ID NO: 4, 5, 10, 11, 16, or 17. A “heterologous” polypeptide, nucleic acid, or gene is one that originates from a different polypeptide, nucleic acid, or gene, or, if from the same polypeptide, nucleic acid, or gene, is substantially modified from its original form.

In another embodiment, the first biological active protein contains the sequence of amino acid residue 3-36 of peptide YY (SEQ ID NO: 19). In this case, the fusion protein can contain the sequence of SEQ ID NO: 12 or 13.

In a further embodiment, the first biological active protein contains the sequence of amino acid residues 1-36 of amylin (SEQ ID NO: 18). For example, the fusion protein contains the sequence of SEQ ID NO: 14 or 15.

The above-discussed fusion protein can further contain a linker segment that joins the first segment and the second segment. The linker segment is capable of dimerizing. It can contain the Fc fragment of an immunoglobulin, e.g., IgA, IgE, IgD, IgG, or IgM, or a functional equivalent there of Preferably, the Fc fragment is that of IgG, which, e.g., contains SEQ ID NO.: 3.

The fusion protein can further contain SEQ ID NO.: 9 or a functional equivalent thereof. SEQ ID NO: 9 is a tPA secretion signal peptide sequence. When fused to the C-terminus of a matured protein or peptide, it directs the protein or peptide to the secretion pathway and the extracellular space (e.g., a culture medium of a cell expressing the protein or peptide). The tPA signal peptide can be cleaved from the mature protein or peptide after the secretion. Similar signal peptides such as those from IgG heavy and light chain can also be used for the same secretion purpose.

Another aspect of the invention features an isolated nucleic acid comprising a sequence that encodes the fusion protein described above. The nucleic acid can contain the sequence of one SEQ ID NOs: 6-7.

A nucleic acid refers to a DNA molecule (e.g., a cDNA or genomic DNA), an RNA molecule (e.g., an mRNA), or a DNA or RNA analog. A DNA or RNA analog can be synthesized from nucleotide analogs. The nucleic acid molecule can be single-stranded or double-stranded, but preferably is double-stranded DNA. An “isolated nucleic acid” refers to a nucleic acid the structure of which is not identical to that of any naturally occurring nucleic acid or to that of any fragment of a naturally occurring genomic nucleic acid. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. The nucleic acid described above can be used to express the fusion protein of this invention. For this purpose, one can operatively linked the nucleic acid to suitable regulatory sequences to generate an expression vector.

A vector refers to a nucleic acid molecule capable of transporting another nucleic acid to which it has been linked. The vector can be capable of autonomous replication or integrate into a host DNA. Examples of the vector include a plasmid, cosmid, or viral vector. The vector includes a nucleic acid in a form suitable for expression of the nucleic acid in a host cell. Preferably the vector includes one or more regulatory sequences operatively linked to the nucleic acid sequence to be expressed. A “regulatory sequence” includes promoters, enhancers, and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein or RNA desired, and the like. The expression vector can be introduced into host cells to produce a polypeptide of this invention. Also within the scope of this invention is a host cell that contains the above-described nucleic acid. Examples include E. coli cells, insect cells (e.g., using baculovirus expression vectors), yeast cells, or mammalian cells. See e.g., Goeddel, (1990) Gene Expression Technology: Methods in Enzymology 185, Academic Press, San Diego, Calif. To produce a polypeptide of this invention, one can culture a host cell in a medium under conditions permitting expression of the polypeptide encoded by a nucleic acid of this invention, and purify the polypeptide from the cultured cell or the medium of the cell. Alternatively, the nucleic acid of this invention can be transcribed and translated in vitro, e.g., using T7 promoter regulatory sequences and T7 polymerase.

A “functional equivalent” of a proteinous factor refers to a polypeptide derivative of the protein e.g., a protein having one or more point mutations, insertions, deletions, truncations, a fusion protein, or a combination thereof. It is at least 70% (e.g., 80%, 90%, 95%, or 100%, or any other number between 70% and 100%, inclusive) identical to the factor and retains substantially the activity of the factor, e.g., an ability to bind to a receptor thereof and trigger the corresponding signal transduction pathway.

The “percent identity” of two amino acid sequences or of two nucleic acids is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

Within the scope of this invention is a composition comprising the aforementioned fusion protein or a nucleic acid encoding the fusion protein. The composition can be a pharmaceutical composition that contains a pharmaceutically acceptable carrier or a food composition that contains a dietarily acceptable carrier. The composition can be used to maintain or reduce body weight of a subject in need thereof by administering to the subject an effective amount of the fusion protein or a nucleic acid encoding the fusion protein. For this purposes, the subject can be concurrently administered the first or the second peptide or protein mentioned above in non-fusion form.

The invention features another pharmaceutical composition that includes (i) Leptin or a functional equivalent; (ii) one of Glucagon-like peptide 1, amylin, peptide YY, or a functional equivalent thereof; and (iii) a pharmaceutically acceptable carrier.

The invention also features a food composition comprising a recombinant lactic acid bacterium that produces and secrets the fusion proteins or the first together with the second peptide or protein.

The above-discussed compositions can be used to treat diabetes or obesity. Thus, within the scope of this invention is a method for treating diabetes or obesity. The method includes administering to a subject in need thereof an effective amount of the fusion protein discussed above or a nucleic acid encoding the fusion protein. The method can include concurrently administering to the subject the first (particularly a long-acting version) or the second peptide or protein that are not fused to each other.

In another aspect, the invention features a method of increasing the half-life of a recombinant therapeutic peptide or protein in a subject. The method includes joining a recombinant protein to a segment containing SEQ ID NO: No 1 or a functional equivalent thereof to form a fusion protein; and determining the half-life of the fusion protein in a subject. The therapeutic peptide or recombinant protein has a therapeutic effect on diabetes or obesity.

The invention also features a method of increasing the efficacy of a recombinant therapeutic peptide or protein in a subject. The method includes joining the recombinant protein to a segment containing SEQ ID NO: 1 or a functional equivalent thereof to form a fusion protein chimera; and determining the efficacy of the fusion protein in a subject. The therapeutic peptide or recombinant protein has a therapeutic effect on diabetes or obesity or both. The fusion of SEQ ID NO: 1 increases the efficacy of the recombinant therapeutic peptide or protein via additive or more than additive or synergy effects. The fusion partners do not interference each other's biological function.

The details of one or more embodiments of the invention are set forth in the description below. Other features, objects, and advantages of the invention will be apparent from the description and from the claims.

### DETAILED DESCRIPTION OF THE INVENTION

This invention is based, as least in part, on the discovery of a novel use of Leptin as a functional fusion partner to extend biological lives and efficacy of anti-diabetes or anti-obesity therapeutic peptides or polypeptides. It was unexpected that Leptin or its functional equivalent, when fused to the C-termini of a number of bioactive peptides, e.g., anti-diabetes peptides, extends the biological lives and efficacy of the bioactive peptides with more than additive action or synergy. Examples of these proteins include Glucagon-like peptide 1, amylin, or peptide YY (PYY), or a functional equivalent.

It was known in the art that N-terminal protein fusion to a bioactive protein often leads to complete activity loss, particularly for large-size protein fusion partners. For example, pro-enzymes and pro-hormones are not active due to the propeptide fusion at their N-termini. These pro-digesting enzymes and pro-hormones become biologically active only until after their propeptides are cleaved off. In addition, large size protein fusion often leads to low expression yield. Unexpectedly, Leptin fused proteins can be produced at commercial production level in mammalian host cells. The fusion does not interfere with the activity of Leptin or a bioactive protein to which it is fused. Also, unexpectedly, Leptin or its functional equivalent not only extends biological lives of the bioactive peptides, but also enhance the activity of each other.

In addition, when GLP-1 or its analogues, PYY, or amylin used together with Leptin (in a fusion protein or not), they have more than additive or synergy effects on body weight through reducing appetite or food intake or others. This was unexpected since use of commercial GLP-1 or recombinant leptin alone did not induce significant weight loss in our animal model (our pilot experiments). Thus the concurrent administration of Leptin and GLP-1 or its analogues, PYY, or amylin can be used in treating obesity or diabetes.

For example, as shown in the examples below, a fusion protein GLP1-Fc-leptin not only maintains GLP-1's glucose lowing activity, but also keeps Leptin's weight loss activity. In addition, it has much longer biological life or longer lasting therapeutic effect than GLP-1 analogue E4 Byetta.

### Leptin

Leptin, e.g., GenBank Accession No. NP_{—}000221, is an adipose-derived hormone, a key nutrient sensor that regulates food intake and body weight. Recombinant leptin is an effective weight loss agent in small animals. However, the leptin treatment of obese humans has been restricted to few subjects that suffer from congenital leptin deficiency. Obviously, leptin itself is not a great human therapeutic agent. On other hand, the regulation of human appetite, food intake and weight loss may be regulated by more than one factor. As described herein, use of leptin as a functional fusion partner to extend biological life of other diabetes-related or weight loss therapeutic agents may have additional therapeutic values.

Leptin to be used in this invention may be selected from recombinant murine or recombinant human protein as set forth in US Patent Application 20030203837 and Zhang et al. (Nature, 1994, 372: 425-432; incorporated herein by reference) or those lacking a glutaminyl residue at position 28 (Zhang et al., supra, at page 428.). One can also use the recombinant human Leptin protein analog as set forth in US Patent Application 20030203837 (SEQ ID NO: 4 therein), which contains (1) an arginine in place of lysine at position 35 and (2) a leucine in place of isoleucine at position 74.

Murine Leptin protein is substantially homologous to the human Leptin, particularly as a mature protein, and, further, particularly at the N-terminus. One may prepare an analog of the recombinant human protein by altering (such as substituting amino acid residues), in the recombinant human sequence, the amino acids which diverge from the murine sequence. Because the recombinant human protein has biological activity in mice, such analog would likely be active in humans. For example, using a human protein having a lysine at residue 35 and an isoleucine at residue 74 according to the numbering of SEQ ID NO: 1, one may substitute with another amino acid one or more of the amino acids at positions 32, 35, 50, 64, 68, 71, 74, 77, 89, 97, 100, 105, 106, 107, 108, 111, 118, 136, 138, 142, and 145. One may select the amino acid at the corresponding position of the murine protein.

Rat Leptin protein (Murakami et al., Biochem. Biophys. Res. Comm. 1995; 209: 944-952) or rhesus monkey Leptin protein (US Patent Application 20030203837) can also be used. These Leptin proteins differ from human Leptin protein at a number of positions. One may substitute with another amino acid one or more of the amino acids at these divergent positions to produce Leptin analogous. Other analogs may be prepared by deleting a part of the protein amino acid sequence. See, e.g., US Patent Application 20030203837, which is incorporated by reference.

### Glucagons-Like Peptide -1

Glucagons-like peptide -1 (GLP-1), e.g., that of GenBank Accession No. P01275, is synthesized in intestinal endocrine cells in 2 principle molecular forms as GLP-1 (7-36) and GLP-1 (7-37). The peptide was first identified following the cloning of DNAs and genes for proglucagon. Initial studies of GLP-1 biological activity utilized the full length N-terminal extended forms of GLP-1 (amino acids 1-37 and 1-36). The large GLP-1 molecules were generally devoid of biological activity. Later, in 1987, it was found that removal of the first 6 amino acids resulted in a shorter version of the GLP-1 molecule with substantially enhanced biological activity.

The majority of circulating biologically active GLP-1 is the GLP-1 (6-36), with lesser amount of the bioactive GLP-1 (7-37) form also detectable. The N-terminal is an important locus for regulation of GLP-1 biological activity since dipeptideyl peptidase (DPP-IV) mediated cleavage at the position 2 alanine leads to degradation of the peptide. GLP-1 analogues with position 2 alanine replaced with glutamine or valine are resist to DPP-IV.

GLP-1 has anti-diabetes and anti-obesity potentially beneficial effects. For example, it delays gastric emptying, which blunts hyperglycemia after meals; curbs appetite; inhibits food intake; and causes beta cell growth. These effects are of great interest to pharmaceutical companies. Amylin Pharmaceuticals is marketing an analogue of GLP-1 called for diabetes and obesity related indications. Novo-Nordisk has developed another long-acting GLP-1. At least five other companies now have GLP-1 analogues under development including human Genome Science's albumin fused GLP-1.

### Peptide YY and Amylin

Besides GLP-1 analogues, peptide YY (e.g., GenBank Accession No. P10082), amylin (e.g., GenBank Accession No. P10997), and many other polypeptides or proteins are also potential “anti-obesity” or “anti diabetes” agents and may be fused to leptin to extend their biological lives and to have additive or more than additive action or synergy as one chimeric molecule. As matter of fact, Amylin Pharmaceuticals is currently marketing amylin (commercial name Smylin) for diabetes and obesity related indications.

As described herein, a number of fusion proteins of Leptin and anti-obesity proteins are generated. Examples of them include a monomer form of GLP-1-3xGly-Leptin (SEQ ID NO:4), a dimmer form of GLP-1-3xGly-IgG1 Fc-leptin (SEQ ID NO:5), (G8- or V8-GLP-)-linker-Leptin (SEQ ID NO:10), a dimmer form of GLP-1 analogues (G8- or V8-GLP-1)-linker-IgG1 Fc-leptin (SEQ ID NO: 11). In addition, peptide YY (3-36)-linker-Leptin (SEQ ID NO: 12), a dimmer form of peptide YY (3-36)-linker-IgG1 Fc-leptin (SEQ ID NO: 13), amylin-linker-Leptin (SEQ ID NO: 14), a dimmer form of amylin-linker-IgG1 Fc-leptin (SEQ ID NO: 15) were also made.

These chimeric therapeutic agents have additional advantages as compared with GLP-1 and leptin alone. For example, the chimeric agents are more stable in vivo than Leptin or GLP-1. Phamarkinetics profile, tissue distribution, side effects, and efficacy of the chimeric molecules are different from that of concurrent use of two individual molecules, namely native or analogues of leptin and GLP-1.

Analogs of Leptin, GLP-1, peptide YY, or amylin (or biologically active fragments thereof) can be used in this invention. The sequence of each analog differs from the wild-type sequence by one or more conservative amino acid substitutions or by one or more non-conservative amino acid substitutions, deletions, or insertions which do not abolish its biological activity. The following table list suitable amino acid substitutions:

TABLE
Conservative Amino Acid Replacements

| For Amino Acid | Code | Replace with any of |
|---|---|---|
| Alanine | A | Gly, Ala, Cys |
| Arginine | R | Lys, Met, Ile, |
| Asparagine | N | Asp, Glu, Gln, |
| Aspartic Acid | D | Asn, Glu, Gln |
| Cysteine | C | Met, Thr |
| Glutamine | Q | Asn, Glu, Asp |
| Glutamic Acid | E | Asp, Asn, Gln |
| Glycine | G | Ala, Pro, |
| Isoleucine | I | Val, Leu, Met |
| Leucine | L | Val, Leu, Met |
| Lysine | K | Arg, Met, Ile |
| Methionine | M | Ile, Leu, Val |
| Phenylalanine | F | Tyr, His, Trp |
| Proline | P | |
| Serine | S | Thr, Met, Cys |
| Threonine | T | Ser, Met, Val |
| Tyrosine | Y | Phe, His |
| Valine | V | Leu, Ile, Met |

The fusion protein of described herein may be derivatized by the attachment of one or more chemical moieties to the protein moiety. The chemically modified derivatives may be further formulated for intraarterial, intraperitoneal, intramuscular, subcutaneous, intravenous, oral, nasal, pulmonary, topical or other routes of administration. Chemical modification of biologically active proteins has been found to provide additional advantages under certain circumstances, such as increasing the stability and circulation time of the therapeutic protein and decreasing immunogenicity. See U.S. Pat. No. 4,179,337, Abuchowski et al., in Enzymes as Drugs. (J. S. Holcerberg and J. Roberts, eds. pp. 367-383 (1981); Francis, Focus on Growth Factors 3: 4-10 (May 1992) (published by Mediscript, Mountview Court, Friern Barnet Lane, London N20, OLD, UK).

Chemical moieties suitable for derivatization may be selected from among various water-soluble polymers. The polymer selected should be water-soluble so that the protein to which it is attached does not precipitate in an aqueous environment. Preferably, for therapeutic use of the end-product preparation, the polymer is pharmaceutically acceptable. One skilled in the art will be able to select the desired polymer based on such considerations as whether the polymer/protein conjugate will be used therapeutically, and if so, the desired dosage, circulation time, resistance to proteolysis, and other considerations. For the present proteins and peptides, the effectiveness of the derivatization may be ascertained by administering the derivative, in the desired form (i.e., by osmotic pump, or, more preferably, by injection or infusion, or, further formulated for oral, pulmonary or nasal delivery, for example), and observing biological effects as described herein.

A water-soluble polymer may be selected from the group consisting of, for example, polyethylene glycol, copolymers of ethylene glycol/propylene glycol, carboxymethylcellulose, dextran, polyvinyl alcohol, polyvinyl pyrolidone, poly-1,3-dioxolane, poly 1,3,6-trioxane, ethylene/maleic anhydride copolymer, polyaminoacids (either homopolymers or random or non-random copolymers), and dextran or poly(n-vinyl pyrolidone) polyethylene glycol, propylene glycol homopolymers, polypropylene oxide/ethylene oxide co-polymers, polyoxyethylated polyols, polystyrenemaleate and polyvinyl alcohol. Polyethylene glycol propionaldenhyde may have advantages in manufacturing due to its stability in water.

Fusion proteins may be further attached to polyaminoacids to increase the circulation half life of the protein. For the present therapeutic or cosmetic purposes, such polyamino acid should be those which do not create neutralizing antigenic response, or other adverse response. Such polyamino acid may be selected from the group consisting of serum album (such as human serum albumin), an antibody or portion thereof (such as an antibody constant region, i.e., the Fc region) or other polyamino acids.

The polymer may be of any molecular weight, and may be branched or unbranched. For polyethylene glycol, the preferred molecular weight is between about 2 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The number of polymer molecules so attached may vary, and one skilled in the art will be able to ascertain the effect on function. One may mono-derivatize, or may provide, for a di-, tri-, tetra- or some combination of derivatization, with the same or different chemical moieties (e.g., polymers, such as different weights of polyethylene glycols). The proportion of polymer molecules to protein (or peptide) molecules will vary, as will their concentrations in the reaction mixture. In general, the optimum ratio (in terms of efficiency of reaction in that there is no excess unreacted protein or polymer) will be determined by factors such as the desired degree of derivatization (e.g., mono, di-, tri-, etc.), the molecular weight of the polymer selected, whether the polymer is branched Cr unbranched, and the reaction conditions.

The chemical moieties should be attached to the protein with consideration of effects on functional or antigenic domains of the protein. There are a number of attachment methods available to those skilled in the art. E.g., EP 0 401 384 herein incorporated by reference (coupling PEG to G-CSF), see also Malik et al., Exp. Hematol. 20: 1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residue. Those having a free carboxyl group may include aspartic acid residues, glutamic acid residues, and the C-terminal amino acid residue. Sulfhydrl groups may also be used as a reactive group for attaching the polyethylene glycol molecule(s). Preferred for therapeutic purposes is attachment at an amino group, such as attachment at the N-terminus or lysine group. Attachment at residues-important for receptor binding should be avoided if receptor binding is desired.

One may specifically desire N-terminally chemically modified protein. Using polyethylene glycol as an illustration of the present compositions, one may select from a variety of polyethylene glycol molecules (by molecular weight, branching, etc.), the proportion of polyethylene glycol molecules to protein molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective N-terminal chemical modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved. For example, one may selectively N-terminally pegylate the protein by performing the reaction at a pH which allows one to take advantage of the pKa differences between the epsilon-amino group of the lysine residues and that of the (amino group of the N-terminal residue of the protein. By such selective derivatization, attachment of a water soluble polymer to a protein is controlled: the conjugation with the polymer takes place predominantly at the N-terminus of the protein and no significant modification of other reactive groups, such as the lysine side chain amino groups, occurs. Using reductive alkylation, the water soluble polymer may be of the type described above, and should have a single reactive aldehyde for coupling to the protein. Polyethylene glycol propionaldehyde, containing a single reactive aldehyde, may be used.

An N-terminally monopegylated derivative is preferred for ease in production of a therapeutic. N-terminal pegylation ensures a homogenous product as, characterization of the product is simplified relative to di-, tri- or other multi pegylated products. The use of the above reductive alkylation process for preparation of an N-terminal product is preferred for ease in commercial manufacturing.

In yet another aspect of the present invention, provided are methods of using pharmaceutical compositions of the proteins, and derivatives. Such pharmaceutical compositions may be for administration by injection, or for oral, pulmonary, nasal, transdermal or other forms of administration. In general, comprehended by the invention are pharmaceutical compositions comprising effective amounts of protein or derivative products of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hylauronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference. The compositions may be prepared in liquid form, or maybe in dried powder, such as lyophilized form. Implantable sustained release formulations are also contemplated, as are transdermal formulations.

Contemplated for use herein are oral solid dosage forms, which are described generally in Remington's Pharmaceutical Sciences, 18th Ed. 1990 (Mack Publishing Co. Easton Pa. 18042) at Chapter 89, which is herein incorporated by reference. Solid dosage forms include tablets, capsules, pills, troches or lozenges, cachets or pellets. Also, liposomal or proteinoid encapsulation may be used to formulate the present compositions (U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given by Marshall, K. In: Modern Pharmaceutics Edited by G. S. Banker and C. T. Rhodes Chapter 10, 1979, herein incorporated by reference. In general, the formulation will include the protein (or analog or derivative), and inert ingredients which allow for protection against the stomach environment, and release of the biologically active material in the intestine.

Also specifically contemplated are oral dosage forms of the above derivatized proteins. Protein may be chemically modified so that oral delivery of the derivative is efficacious. Generally, the chemical modification contemplated is the attachment of at least one moiety to the protein (or peptide) molecule itself, where said moiety permits (a) inhibition of proteolysis and (b) uptake into the blood stream from the stomach or intestine. Also desired is the increase in overall stability of the protein and increase in circulation time in the body. Examples of such moieties include: Polyethylene glycol, copolymers of ethylene glycol and propylene glycol, carboxymethyl cellulose, dextran, polyvinyl alcohol, polyvinyl pyrrolidone and polyproline. Abuchowski and Davis, Soluble Polymer-Enzyme Adducts. In: “Enzymes as Drugs”, Hocenberg and Roberts, eds., Wiley-Interscience, New York, N.Y., (1981), pp 367-383; Newmark, et al., J. Appl. Biochem. 4: 185-189 (1982). Other polymers that could be used are poly-1,3-dioxolane and poly-1,3,6-tioxocane.

For the protein (or derivative) the location of release may be-the stomach, the small intestine (the duodenum, the jejunem, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the protein (or derivative) or by release of the biologically active material beyond the stomach environment, such as in the intestine.

To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of dry therapeutic i.e. powder; for liquid forms, a soft gelatin shell may be used. The shell material of cachets could-be thick starch or other edible paper. For pills, lozenges, molded tablets or tablet triturates, moist massing techniques can be used.

The therapeutic can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the protein (or derivative) may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, .alpha.-lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An antifrictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives which potentially enhance uptake of the protein (or derivative) are for instance the fatty acids oleic acid, linoleic acid and linolenic acid.

Controlled release formulation may be desirable. The drug could be incorporated into an inert matrix which permits release by either diffusion or leaching mechanisms i.e. gums. Slowly degenerating matrices may also be incorporated into the formulation. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects. Some entric coatings also have a delayed release effect.

Other coatings may be used for the formulation. These include a variety of sugars which could be applied in a coating pan. A therapeutic agent could also be given in a film coated tablet and the materials used in this instance are divided into 2 groups. The first are the nonenteric materials and include methyl cellulose ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials sight may be used to provide the optimum film coating. Film coating may be carried out in a pan coater or in a fluidized bed or by compression coating.

Also contemplated herein is a novel oral delivery system of the present protein, or derivative thereof through a food-grade lactic acid bacteria expression system. A gene encoding a fusion protein can be reconstructed into food-grade expression plasmid pLEB590 and pLEB600 (Timo Takala, PhD thesis, ISBN 952-10-2260-4; available at http://ethesis.helsinki.fi) where an effective secretion leader sequence such as usp45 is incorporated at its N-terminus. The reconstructed plasmid may be further transferred into food-grade lactic acid bacteria for expression and proliferation. The transformed lactic acid bacteria expressing secreted fusion protein such as GLP1-leptin can be freeze-dried for oral delivery. The lactic acid bacteria are acid-resistant and may easily pass the stomach low pH barrier and may stay in intestine for days. The secreted fusion proteins may be absorbed into intestine directly for efficacy.

Also contemplated herein is pulmonary delivery of the present protein, or derivative thereof A fusion protein is delivered to the lungs of a mammal while inhaling and traverses across the lung epithelial lining to the blood stream. See, e.g., Adjei et al., Pharmaceutical Research 1990, 7: 565-569; Adjei et al., International Journal of Pharmaceutics 1990, 63: 135-144; Braquet et al., Journal of Cardiovascular pharmacology 1989, 13 (suppl. 5): s.143-146; Hubbard et al., Annals of Internal Medicine 1989, 3: 206-212; Smith et al., J. Clin. Invest. 1989, 84: 1145-1146; Oswein et al. “Aerosolization of Proteins”, Proceedings of Symposium on Respiratory Drug Delivery II, Keystone, Colo., 1990, March; Debs et al., The Journal of Immunology 1998, 140: 3482-3488, and U.S. Pat. No. 5,284,656.

Contemplated for use in the practice of this invention are a wide range of mechanical devices designed for pulmonary delivery of therapeutic produces, including but not limited to nebulizers, metered dose inhalers, and powder inhalers all of-which are familiar to those skilled in the art.

Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of protein (or analog or derivative). Typically, each formulation is specific to the type of device employed and may involve the use of an appropriate propellant material, in addition to diluents, adjuvants and/or carriers useful in therapy.

The protein (or derivative) should most advantageously be prepared in particulate form with an average particle size of less than 10 μm (or microns), most preferably 0.5 to 5 μm, for most effective delivery to the distal lung. Carriers include carbohydrates such as trehalose, mannitol, xylitol, sucrose, lactose, and sorbitol. Other ingredients for use in formulations ray include DPPC, DOPE, DSPC and DOPC. Natural or synthetic surfactants may be used. Polyethylene glycol may be used (even apart from its use in derivatizing the protein or analog). Dextrans, such as cyclodextran, may be used. Bile salts and other related enhancers may be used. Cellulose and cellulose derivatives may be used. Amino acids may be used, such as use in a buffer formulation.

Also, the use of liposomes, microcapsules or microspheres, inclusion complexes, or other types of carriers is contemplated.

Formulations suitable for use with a nebulizer, either jet or ultrasonic, will typically comprise protein (or derivative) dissolved in water at a concentration of about 0.1 to 25 mg of biologically active protein per mL of solution. The formulation may also include a buffer and a simple sugar (e.g., for protein stabilization and regulation of osmotic pressure). The nebulizer formulation may also contain a surfactant, to reduce or prevent surface induced aggregation of the protein caused by atomization of the solution in forming the aerosol.

Formulations for use with a metered-dose inhaler device will generally comprise a finely divided powder containing the protein (or derivative) suspended in a propellant with the aid of a surfactant. The propellant may be any conventional material employed for this purpose, such as a chlorofluorocarbon, a hydrochlorofluorocarbon, a hydrofluorbcarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing protein (or derivative) and may also include a bulking agent, such as lactose, sorbitol, sucrose, mannitol, trehalose, or xylitol in amounts which facilitate dispersal of the powder from the device, e.g., 50 to 90% by weight of the formulation.

Nasal delivery of the protein (or analog or derivative) is also contemplated. Nasal delivery allows the passage of the protein to the blood stream directly after administering the therapeutic product to the nose, without the necessity for deposition of the product in the lung. Formulations for nasal delivery include those with dextran or cyclodextran. Delivery via transport across other mucus membranes is also contemplated.

Within the scope of this invention is a method of treating diabetes or obesity by administering to a subject in need thereof an effective amount of the fusion protein of this invention. Subjects to be treated can be identified as having or being at risk for acquiring a condition characterized by diabetes or obesity. This method can be performed alone or in conjunction with other drugs or therapy. The term “treating” refers to administration of a composition to a subject with the purpose to cure, alleviate, relieve, remedy, prevent, or ameliorate a disorder, the symptom of the disorder, the disease state secondary to the disorder, or the predisposition toward the disorder. An “effective amount” is an amount of the composition that is capable of producing a medically desirable result in a treated subject. The medically desirable result may be objective (i.e., measurable by some test or marker) or subjective (i.e., subject gives an indication of or feels an effect).

A subject to be treated may be identified as being in need of treatment for one or more of the disorders noted above. Identifying a subject in need of such treatment can be in the judgment of a subject or a health care professional, and can be subjective (e.g., opinion) or objective (e.g., measurable by a test or diagnostic method).

In one in vivo approach, a therapeutic composition (e.g., a composition containing a fusion protein of the invention) is administered to the subject. Generally, the protein is suspended in a pharmaceutically-acceptable carrier (e.g., physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The dosage required depends on the choice of the route of administration; the nature of the formulation; the nature of the subject's illness; the subject's size, weight, surface area, age, and sex; other drugs being administered; and the judgment of the attending physician. Suitable dosages are in the range of 0.01-100.0 mg/kg. Variations in the needed dosage are to be expected in view of the variety of compositions available and the different efficiencies of various routes of administration. For example, oral administration would be expected to require higher dosages than administration by intravenous injection. Variations in these dosage levels can be adjusted using standard empirical routines for optimization as is well understood in the art. Encapsulation of the composition in a suitable delivery vehicle (e.g., polymeric microparticles or implantable devices) may increase the efficiency of delivery, particularly for oral delivery.

The efficacy of a composition of this invention can be evaluated both in vitro and in vivo. See, e.g., the examples below. Briefly, the composition can be tested for its efficacy in vitro. For in vivo studies, the composition can be injected into an animal (e.g., a mouse model) and its therapeutic effects are then accessed. Based on the results, an appropriate dosage range and administration route can be determined.

The present methods may be used in conjunction with other medicaments, such as those useful for the treatment of diabetes. (e.g., insulin, and possibly amylin), cholesterol and blood pressure lowering medicaments (such as those which reduce blood lipid levels or other cardiovascular medicaments), and activity increasing medicaments (e.g., amphetamines). Appetite suppressants may also be used. Such administration may be simultaneous or may be in seriatim.

In addition, the present methods may be used in conjunction with surgical procedures, such as cosmetic surgeries designed to alter the overall appearance of a body (e.g., liposuction or laser surgeries designed to reduce body mass, or implant surgeries designed to increase the appearance of body mass). The health benefits of cardiac surgeries, such as bypass surgeries or other surgeries designed to relieve a deleterious condition caused by blockage of blood vessels by fatty deposits, such as arterial plaque, may be increased with concomitant use of the present compositions and methods. Methods to eliminate gall stones, such as ultrasonic or laser methods, may also be used either prior to, during or after a course of the present therapeutic methods.

The examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### Example 1

Constructs encoding fusion proteins GLP-1-Fc-leptin and GLP-1-3G-leptin were prepared.

Specifically, EcorRI-tPA-GLP-1-3xGly-leptin-Not I cDNA was synthesized first by a commercial service provider (Genscript) and digested with EcoRI ad Not I. The resulting fusion sequence (SEQ ID NO:6) was then cloned into a CMV-based mammalian expression vector pCA, pCApuro and pCAdhfr for mammalian expression.

To construct an expressing vector encoding GLP-1-3xGly-IgG1 Fc-leptin (SEQ ID NO:5), a standard multiple-step PCR method was used to generate the coding sequence (SEQ ID NO: 7). In brief, the above fusion sequence (SEQ ID NO:6) and IgG1 Fc cDNA (SEQ ID NO:8) were used as PCR templates. Primers were designed to synthesize three overlapping fragments of GLP-1, IgG1 Fc, and leptin. These fragments were combined to make final sequence (SEQ ID NO: 7) by a 2-step PCR reactions. The resulting fusion sequence included a Kozac sequence, a tPA signal sequence at its N-terminus GLP-1-3xGly-IgG1 Fc-leptin cDNA. This sequence was ligated into the CMV-based mammalian expression vector pCApuro and pCAdhfr for mammalian expression.

### Example 2

Fusion proteins GLP-1-3xGly-IgG Fc-leptin and GLP-1-3G-leptin were expressed in CHO cells that were cultured in a serum-free suspension. The above described two constructs were expressed in a CHO cell line by a standard method. The tPA secretion signal (SEQ ID NO: 9) directed the expressed fusion protein in the cultured medium. The culture medium from each cell clone was collected and subjected to dot blot analysis using rabbit anti human Fc fragment antibodies (PIERCE, Product# 0031423). It was found a number of cell clones express high levels of fusion proteins.

### Example 3

GLP-1-3xGly-IgG Fc-leptin was scaled up in serum-free suspension culture. Expression titers and robustness of clones expressing GLP-1-3xGly-IgG Fc-leptin (SEQ ID NO:5) were conducted in serum-free animal component-free medium in 96-well plate and followed by 125 ml shaker flask fedbatch studies. The clones having high expressing levels were scaled up in a 4 liter suspension culture vessel containing serum-free animal component-free medium. Expression titer in the conditional medium was studied by dot blot in the manner described above. It was found that scaling up was successful.

To produce the fusion protein, media were collected and filtered. The protein was purified by using protein-A affinity resin (Repligen) and eluted by 0.5 M arginine HCl pH 3.3 buffer. The purified bulk was formulated in a buffer containing 1% arginine HCl, 5 mM histidine, 0.1% Tween-20 and 1% mannitol at pH5.0 and stored at −80 C.

The following molecules were also constructed and expressed in a manner similar to that described above. These molecules (1) modified GLP-1 (G8-GLP-)-linker (GGGSGGGS)-Leptin (SEQ ID NO: 10); (2) the dimmer form of modified GLP-1 (G8-GLP-1)-linker (GGGGSGGGGS)-IgG1 Fc-leptin (SEQ ID NO:11); (3) the peptide YY (3-36)-linker-Leptin (SEQ ID NO: 12); (4) the dimmer form of peptide YY (3-36)-linker-IgG1 Fc-leptin (SEQ ID NO:13); (5) the amylin-linker-leptin (SEQ ID NO: 14); (6) the dimmer form of amylin-linker-IgG1 Fc-leptin (SEQ ID NO: 15); (7) the monomer form of G8-GLP1-3Gly-leptin (SEQ ID NO: 16); and (8) the dimmer form of G8-GLP1-3xGly-IgG1 Fc-leptin (SEQ ID NO:17).

### Example 4

GLP-1-3xGly-IgG Fc-leptin was purified. The above-described media were filtered and purified using protein-A affinity column for binding (Regeneron) and 0.5 M arginine-HCl at pH3.5 for elution. The purified protein was studied by SDS-page gel.

### Example 5

The expressed GLP-1-3xGly-IgG Fc-leptin was characterized. The molecular integrity of expressed protein was determined by reduced and non-reduced Western blot using HRP-conjugated rabbit anti human IgG1 Fc (PIERCE, Product# 0031423), goat anti-human leptin antibodies (R&D systems, Cat# AF398), and HRP-conjugated bovine anti goat IgG antibodies (Santa Cruz biotechnology Inc, Cat# sc-2350), rabbit anti GLP-1 antibodies (Alpha diagnostic International, Cat# GLP15-P), and HRP-conjugated goat anti rabbit IgG antibodies (Santa Cruz Biotechnology Inc, Cat # sc-2004). It was found that the expressed GLP-1-3xGly-IgG Fc-leptin was recognized by all the primary antibodies. The results demonstrate that in tact GLP-1-3xGly-IgG Fc-leptin were expressed.

### Example 6

Therapeutic activity of GLP-1-3xGly-IgG Fc-leptin was studied.

First, intraperitoneal glucose tolerance test (ip GTT) was conducted to test GLP -Fc-leptin's action on glucose-dependent insulin secretion. Briefly, mouse blood glucose was measured using one-touch blood glucose strips. A blood sample was obtained from a mouse through tail bleeding. Then, 0.04, 0.1, or 0.2 mg of GLP1-Fc-leptin or control protein human IgG1 Fc fragment were injected ip. Immediately after the injections, 0.2 ml of saturated glucose water solution was injected ip into the mouse. One and two hours later, another blood sample was collected in the same manner for glucose measurement. Byetta (0.025 mg; commercial name of GLP-1 analogue E4, Amylin Pharmaceuticals Inc) was used as positive control. The results are summarized in Tables 1-4 below:

TABLE 1
Effects of Byetta on blood glucose level

| | Byetta Blood glucose | H2O Blood glucose |
|---|---|---|
| | (X ± SD)(n = 5) | (X ± SD)(n = 5) |
| Before IP glucose | 89.4 ± 21.9 | 96.4 ± 17.9 |
| injection | | |
| 1 hour after IP glucose | 127.6 ± 70.4 | 320.8 ± 177.3 |
| injection | | |
| 2 hour after IP glucose | 68.6 ± 26.6 | 125.2 ± 20.7 |
| injection | | |

TABLE 2
Effects of 0.04 mg GLP1-Fc-leptin on blood glucose level.

| | GLP1-Fc-leptin | Fc control |
|---|---|---|
| | Blood glucose | Blood glucose |
| IP glucose tolerance test | (X ± SD)(n = 5) | (X ± SD)(n = 5) |
| Before IP glucose injection | 98.8 ± 29.2 | 88.3 ± 24.5 |
| 1 hour after IP glucose | 275.8 ± 154.0 | 254.0 ± 107.5 |
| injection | | |
| 2 hour after IP glucose | 98.0 ± 22.7 | 94.5 ± 22.3 |
| injection | | |

TABLE 3
Effects of 0.1 mg GLP1-Fc-leptin on blood glucose level.

| | GLP1-Fc-leptin | Fc control |
|---|---|---|
| | Blood glucose | Blood glucose |
| IP glucose tolerance test | (X ± SD)(n = 4) | (X ± SD)(n = 4) |
| Before IP glucose injection | 67.3 ± 32.1 | 51.5 ± 30.4 |
| 1 hour after IP glucose | 108.8 ± 44.0 | 203.3 ± 143.4 |
| injection | | |
| 2 hour after IP glucose | 83.0 ± 21.6 | 122.3 ± 43.8 |
| injection | | |

TABLE 4
Effects of 0.2 mg GLP1-Fc-leptin on blood glucose level.

| | GLP1-Fc-leptin | Fc control |
|---|---|---|
| | Blood glucose | Blood glucose |
| IP glucose tolerance test | (X ± SD)(n = 5) | (X ± SD)(n = 5) |
| Before IP glucose injection | 84.4 ± 20.5 | 76.6 ± 24.8 |
| 1 hour after IP glucose | 113.0 ± 16.2 | 255.2 ± 205.2 |
| injection | | |
| 2 hour after IP glucose | 82.0 ± 6.2 | 174.4 ± 112.1 |
| injection | | |

As shown in Table 1, injections of Byetta, 0.1 mg GLP1-Fc-leptin, and 0.2 mg GLP1-Fc-leptin significantly inhibited blood glucose levels (P<0.01, 0.05, and 0.05, respectively.). The above results demonstrate that GLP1-Fc-leptin can be used to decrease blood glucose levels in a dose-dependent manner.

The above experiment was repeated with GLP1-Fc-leptin (0.2 mg) at day-1 before the glucose tolerance test. A IP glucose tolerance test was conducted twice at day-1 and day-2 respectively. The results are summarized in Table 5 below:

TABLE 5
Long-term Effects of 0.2 mg GLP1-Fc-leptin
on blood glucose level.

| | Day-1 GLP1-Fc- | Day-2 GLP1-Fc- | |
|---|---|---|---|
| | leptin | leptin | Day-1 Fc control |
| IP glucose | Blood glucose | Blood glucose | Blood glucose |
| tolerance test | (X ± SD)(n = 5) | (X ± SD)(n = 5) | (X ± SD)(n = 5) |
| Before IP | 84.4 ± 20.5 | 92.6 ± 25.3 | 76.6 ± 24.8 |
| glucose | | | |
| injection | | | |
| 1 hour after IP | 113.0 ± 16.2 | 106.2 ± 25.7 | 255.2 ± 205.2 |
| glucose | | | |
| injection | | | |
| 2 hour after IP | 82.0 ± 6.2 | 95.8 ± 21.1 | 174.4 ± 112.1 |
| glucose | | | |
| injection | | | |

As shown in Table-5, one injection of 0.2 mg GLP1-Fc-leptin inhibited blood glucose level and last for at least two days.

The effects of GLP1-Fc-leptin on body weight were studied. Mice were injected with 0.1 mg of GLP1-Fc-leptin or human IgG1 Fc fragment in the same manner described above daily for seven days. At days 1 and 7, the body weight of each rat was measured and recorded. The results are summarized in Table 6 below.

TABLE 6
Effects of GLP1-Fc-leptin on body weight

| | GLP1-Fc-leptin | Fc control |
|---|---|---|
| | Body weight | Body weight |
| Day | (X ± SD)(n = 5) | (X ± SD)(n = 5) |
| 1 | 24.7 ± 1.3 | 23.5 ± 2.7 |
| 4 | 23.7 ± 2.2 | 23.7 ± 3.0 |
| 7 | 21.9 ± 1.4 | 23.7 ± 3.2 |

It was found, at day 7, mice injected with GLP1-Fc-leptin lost 11.3% body weight (p<0.05). In contrast, no body weight loss was observed in mice injected with human IgG1 Fc fragment. These results demonstrate that GLP1-Fc-leptin can be used to reduce body weight.

Next, effects of Byetta (GLP-1 analogue E4) on body weight were studied in the same manner. Five microgram Byetta was injected into each mouse twice daily for 7 days. The body weight was measured on days 1, 4, and 7. The results are summarized in Table 7 below.

TABLE 7
Effects of Byetta on Body weight

| | Byetta | Fc control |
|---|---|---|
| | Body weight | Body weight |
| Day | (X ± SD)(n = 5) | (X ± SD)(n = 5) |
| 1 | 21.6 ± 2.0 | 21.3 ± 3.7 |
| 4 | 21.1 ± 2.2 | 21.4 ± 3.3 |
| 7 | 21.3 ± 2.2 | 22.2 ± 3.9 |

It was found that the administration of Byetta had no statistically significant effects on body weight as compared with IgG1 Fc fragment.

In the above experiments, during the 7-day period, blood glucose levels of each mouse were measured and recorded on each day one hour after injection of GLP1-Fc-leptin (0.1 mg) or IgG1 Fc fragment everyday in the morning. The same test was performed using Byetta (GLP-1 analogue 5 ug; twice a day). The results are summarized in Tables 8 and 9 below

TABLE 8
Effects GLP1-Fc-Leptin on Everyday Blood Glucose Level

| | GLP1-Fc-leptin | Fc Control |
|---|---|---|
| | Glucose | glucose |
| Day | mg/dl (n = 5) | mg/dl (n = 5) |
| 1 | 98.0 ± 13.2 | 94.0 ± 19.0 |
| 2 | 119.0 ± 25.8 | 126.0 ± 24.9 |
| 3 | 91.4 ± 44.6 | 126.6 ± 27.8 |
| 4 | 116.0 ± 34.3 | 134.6 ± 48.6 |
| 5 | 95.6 ± 33.9 | 128.4 ± 25.2 |
| 6 | 102.3 ± 25.7 | 126.0 ± 25.5 |
| 7 | 160.4 ± 18.6 | 180.8 ± 15.6 |

TABLE 8
Effects Byetta on Everyday Blood Glucose Level

| | Byetta | Fc Control |
|---|---|---|
| | Glucose | glucose |
| Day | mg/dl (n = 5) | mg/dl (n = 5) |
| 1 | 88.8 ± 4.8 | 128.0 ± 25.4 |
| 2 | 87.4 ± 15.6 | 136.6 ± 27.7 |
| 3 | 93.8 ± 12.4 | 172.0 ± 49.3 |
| 4 | 85.6 ± 11.5 | 126.6 ± 29.7 |
| 5 | 89.8 ± 9.4 | 136.0 ± 19.1 |
| 6 | 106.4 ± 13.2 | 135.8 ± 18.4 |
| 7 | 103.0 ± 18.2 | 137.2 ± 25.6 |

As shown in Tables 8 and 9 below, mice injected with GLP1-Fc-leptin or Byetta had lower blood glucose levels than those injected with IgG1 Fc fragment.

Effects of leptin on body weight were studied in the same manner described above. More specifically, mice were injected ip. with 0.1 mg/mouse of human recombinant leptin (R&D Systems, Cat# 398-LP) or human IgG Fc twice a day (9 am and 5 pm) for 7 days. The results are summarized in Table 10 below.

TABLE 7
Effects of Leptin on Body weight

| | Human leptin | Fc control |
|---|---|---|
| | Body weight | Body weight |
| Day | (X ± SD)(n = 5) | (X ± SD)(n = 5) |
| 1 | 20.9 ± 2.0 | 21.3 ± 3.7 |
| 4 | 20.7 ± 2.3 | 21.4 ± 3.3 |
| 7 | 20.1 ± 2.1 | 22.2 ± 3.9 |

As shown in Table 10, Leptin resulted in less than half of the weight loss that GLP1-Fc-Leptin induced.

Similarly GTT assays were also conducted in rats and rabbits in small numbers. All the results support the inhibition of GLP1-Fc-leptin on blood glucose level.

In summary, GLP1-Fc-leptin not only maintains GLP-1's glucose lowing activity, but also keeps leptin's weight loss effect when comparing with commercial GLP-1 analogue E4 Byetta. In addition, GLP1-Fc-leptin has a much longer lasting therapeutic effect than GLP-1 analogue E4 Byetta Thus, for clinic use, much less injection frequency is required. Also, commercial GLP-1 analogue E4 Byetta (Table 7) or recombinant leptin (Table 10) ip injection alone or combined their effect together did not result in similar degree of the weight loss that GLP1-Fc-Leptin has induced. In conclusion, use GLP-1 together with leptin (e.g., as a fusion protein) has a more than additive or synergetic effect on weight loss.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What we claim is:

1. A fusion protein comprising
a first segment that is located at the amino terminus of the fusion protein and contains the sequence of a first biological active peptide or protein; and
a second segment that is located at the carboxyl terminus of the fusion protein and contains the sequence of a second biological active protein or peptide wherein the first and second segments are operably and covalently linked.

2. The fusion protein of claim 1, wherein the first or second biological active peptide or protein is a peptide or protein hormone.

3. The fusion protein of claim 2, wherein the first biological active protein contains the sequence of Glucagon-like peptide 1, amylin, or peptide YY, or a functional equivalent thereof.

4. The fusion protein of claim 3, wherein the first biological active protein contains the sequence of SEQ ID NO: 2.

5. The fusion protein of claim 2, wherein the second biological active protein contains the sequence of Leptin or a functional equivalent or a weight loss related protein, wherein the second biological active protein maintains a function thereof when covalently fused to the C-terminus of a heterologous peptide or protein.

6. The fusion protein of claim 5, wherein the second biological active protein contains the sequence of SEQ ID NO: 1.

7. The fusion protein of claim 6, wherein the fusion protein contains the sequence of SEQ ID NO: 4, 5, 10, 11, 16, or 17.

8. The fusion protein of claim 3, wherein the first biological active protein contains the sequence of amino acid residue 3-36 of peptide YY of SEQ ID NO:19.

9. The fusion protein of claim 8, wherein the fusion protein contains the sequence of SEQ ID NO: 12 or 13.

10. The fusion protein of claim 3, wherein the first biological active protein contains the sequence of amino acid residues 1-36 of amylin of SEQ ID NO: 18.

11. The fusion protein of claim 10, wherein the fusion protein contains the sequence of SEQ ID NO: 14 or 15.

12. The fusion protein of claim 1, further comprising a linker segment that joins the first segment and the second segment, wherein the linker segment is capable of dimerizing.

13. The fusion protein of claim 12, wherein the linker segment contains the Fc fragment of an immunoglobulin or a functional equivalent there of.

14. The fusion protein of claim 13, wherein the immunoglobulin is IgA, IgE, IgD, IgG, or IgM.

15. The fusion protein of claim 14, wherein the immunoglobulin is IgG.

16. The fusion protein of claim 15, wherein the Fc fragment contains SEQ ID NO.: 3.

17. The fusion protein of claim 1, wherein the fusion protein further contains SEQ ID NO.: 9 or a functional equivalent thereof before the secretion.

18. An isolated nucleic acid comprising a sequence that encodes the fusion protein of claim 1.

19. The nucleic acid of claim 18, wherein the nucleic acid contains the sequence of one of SEQ ID NOs: 6-8.

20. A vector comprising the nucleic acid of claim 18.

21. A host cell comprising a nucleic acid of claim 18.

22. A method of producing a polypeptide, comprising culturing the host cell of claim 21 in a medium under conditions permitting expression of a polypeptide encoded by the nucleic acid, and purifying the polypeptide from the cultured cell or the medium of the cell.

23. A pharmaceutical composition comprising the fusion protein of claim 1 or a nucleic acid encoding the fusion protein; and a pharmaceutically acceptable carrier.

24. A food composition comprising the fusion protein of claim 1 or a nucleic acid encoding the fusion protein; and a dietarily acceptable carrier.

25. A method for reducing body weight, comprising administering to a subject in need thereof an effective amount of the fusion protein of claim 1 or a nucleic acid encoding the fusion protein.

26. The method of claim 25, further comprising concurrently administering to the subject the first or the second peptide or protein.

27. A method for treating diabetes, comprising administering to a subject in need thereof an effective amount of the fusion protein of claim 1 or a nucleic acid encoding the fusion protein.

28. The method of claim 27, further comprising concurrently administering to the subject the first or the second peptide or protein.

29. A method of increasing the half life of a recombinant therapeutic peptide or protein in a subject, the method comprising:
joining a recombinant therapeutic protein to a segment containing SEQ ID NO: 1 or a functional equivalent thereof to form a fusion protein; and
determining the half-life of the fusion protein in a subject,

30. The method of claim 29, wherein the therapeutic recombinant peptide or protein has a therapeutic effect on diabetes or obesity.

31. A method of increasing the efficacy of a therapeutic peptide or protein, comprising:
joining the therapeutic peptide or recombinant protein to a segment containing SEQ ID NO: 1 or a functional equivalent there of to form a fusion protein chimera; and
determining the efficacy of the fusion protein in a subject.

32. The method of claim 31, wherein the therapeutic peptide or recombinant protein has a therapeutic effect on diabetes or obesity.

33. A pharmaceutical composition comprising (i) Leptin or a functional equivalent; (ii) one of Glucagon-like peptide 1, amylin, peptide YY, or a functional equivalent thereof; and (iii) a pharmaceutically acceptable carrier.

34. A food composition comprising (i) Leptin or a functional equivalent; (ii) one of Glucagon-like peptide 1, amylin, peptide YY, or a functional equivalent thereof; and (iii) a dietarily acceptable carrier.

35. A method for treating diabetes or reducing body weight, comprising administering to a subject in need thereof an effective amount of the pharmaceutical composition of claim 33.

36. A method for treating diabetes or reducing body weight, comprising administering to a subject in need thereof an effective amount of the pharmaceutical composition of claim 33.

37. A food composition comprising a recombinant lactic acid bacterium that produces and secrets the fusion protein of claim 1 or a functional equivalents; and a dietarily acceptable carrier.

38. A food composition comprising a recombinant lactic acid bacterium that produces and secrets the first or a long-acting version of the first together with the second or a long-acting version of the second.

39. A food composition comprising a recombinant lactic acid bacterium that produces and secrets (i) Leptin or a functional equivalent together with (ii) one of Glucagon-like peptide 1, amylin, peptide YY, or a functional equivalent thereof.


SEQUENCE LISTING (21 sequences)

SEQ ID NO: 1 - protein, 146 aa, Homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 2 - protein, 30 aa, Homo sapiens
HAEGTFTSDVSSYLEGQAAKEFIAWLVKGR

SEQ ID NO: 3 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - protein, 179 aa, Artificial Sequence
  Description of Artificial Sequence Synthetic construct
HAEGTFTSDVSSYLEGQAAKEFIAWLVKGRGGGVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 5 - protein, 405 aa, Artificial Sequence
  Description of Artificial Sequence Synthetic construct
HAEGTFTSDVSSYLEGQAAKEFIAWLVKGRGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 6 - DNA, 612 bp, Artificial Sequence
  Description of Artificial Sequence Synthetic construct
atggatgcaatgaagagagggctctgctgtgtgctgctgctgtgtggagcagtcttcgtttcgaacagccatgctgaagggacctttaccagtgatgtaagttcttatttggaaggccaagctgccaaggagttcattgcttggctggtgaaaggccgaggcggcggcgtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctgataa

SEQ ID NO: 7 - DNA, 1290 bp, Artificial Sequence
  Description of Artificial Sequence Synthetic construct
atggatgcaatgaagagagggctctgctgtgtgctgctgctgtgtggagcagtcttcgtttcgaacagccatgctgaagggacctttaccagtgatgtaagttcttatttggaaggccaagctgccaaggagttcattgcttggctggtgaaaggccgaggcggcggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctga

SEQ ID NO: 8 - DNA, 681 bp, Homo sapiens
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 9 - protein, 23 aa, Artificial Sequence
  Description of Artificial Sequence Synthetic peptide
MDAMKRGLCCVLLLCGAVFVSNS

SEQ ID NO: 10 - protein, 186 aa, Artificial Sequence
  Description of Artificial Sequence Synthetic construct
HGEGTFTSDVSSYLEGQAAKEFIAWLVKGRGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 11 - protein, 412 aa, Artificial Sequence
  Description of Artificial Sequence Synthetic construct
HGEGTFTSDVSSYLEGQAAKEFIAWLVKGRGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 12 - protein, 190 aa, Artificial Sequence
  Description of Artificial Sequence Synthetic construct
IKPEAPGEDASPEELNRYYASLRHYLNLVTRQRYGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 13 - protein, 416 aa, Artificial Sequence
  Description of Artificial Sequence Synthetic construct
IKPEAPGEDASPEELNRYYASLRHYLNLVTRQRYGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 14 - protein, 193 aa, Artificial Sequence
  Description of Artificial Sequence Synthetic construct
KCNTATCATGRLANFLVHSSNNFGAILSSTNVGSNTYGGGGSGGGGSVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 15 - protein, 419 aa, Artificial Sequence [sequence omitted]

SEQ ID NO: 16 - protein, 179 aa, Artificial Sequence
  Description of Artificial Sequence Synthetic construct
HGEGTFTSDVSSYLEGQAAKEFIAWLVKGRGGGVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 17 - protein, 412 aa, Artificial Sequence; identical to SEQ ID NO: 11
  Description of Artificial Sequence Synthetic construct

SEQ ID NO: 18 - protein, 37 aa, Homo sapiens
KCNTATCATGRLANFLVHSSNNFGAILSSTNVGSNTY

SEQ ID NO: 19 - protein, 34 aa, Homo sapiens
IKPEAPGEDASPEELNRYYASLRHYLNLVTRQRY

SEQ ID NO: 20 - protein, 8 aa, Artificial Sequence
  Description of Artificial Sequence Synthetic peptide
GGGSGGGS

SEQ ID NO: 21 - protein, 10 aa, Artificial Sequence
  Description of Artificial Sequence Synthetic peptide
GGGGSGGGGS